(19) United States
(12) Patent Application Publication
(10) Pub. No.: US 2004/0105816 A1
(43) Pub. Date: Jun. 3, 2004

(54) Cancerous disease modifying antibodies

(76) Inventors: David S. F. Young, Toronto (CA); Helen P. Findlay, Toronto (CA); Susan E. Hahn, Toronto (CA)

Correspondence Address: Michael A. Slavin, McHale & Slavin, P.A., 2855 PGA Boulevard, Palm Beach Gardens, FL, 33410, US

(21) Appl. No.: 10/603,006

(22) Filed: Jun. 23, 2003

Related U.S. Application Data
(63) Continuation-in-part of application No. 10/348,284, filed on Jan. 21, 2003.
Application No. 10/348,284 is a continuation-in-part of application No. 09/415,278, filed on Oct. 8, 1999, now Pat. No. 6,180,357.

(51) Int. Cl. A61K 51/00; A61K 39/395

(52) U.S. Cl. 424/1.49; 424/155.1

(57) ABSTRACT

The present invention relates to a method for producing patient cancerous disease modifying antibodies using a novel paradigm of screening. By segregating the anti-cancer antibodies using cancer cell cytotoxicity as an end point, the process makes possible the production of anti-cancer antibodies for therapeutic and diagnostic purposes. The antibodies can be used in aid of staging and diagnosis of a cancer, and can be used to treat primary tumors and tumor metastases. The anti-cancer antibodies can be conjugated to toxins, enzymes, radioactive compounds, and hematogenous cells.

### REFERENCE TO RELATED APPLICATIONS

This application is a continuation-in-part of application Ser. No. 10/348,284, filed Jan. 20, 2003, which is a continuation-in-part of application Ser. No. 09/415,278, filed Oct. 8, 1999, now U.S. Pat. No. 6,180,357 B1, the contents of each of which are herein incorporated by reference.

### FIELD OF THE INVENTION

This invention relates to the isolation and production of cancerous disease modifying antibodies (CDMAB) and to the use of these CDMAB in therapeutic and diagnostic processes, optionally in combination with one or more chemotherapeutic agents. The invention further relates to binding assays which utilize the CDMAB of the instant invention.

### BACKGROUND OF THE INVENTION

Each individual who presents with cancer is unique and has a cancer that is as different from other cancers as that person's identity. Despite this, current therapy treats all patients with the same type of cancer, at the same stage, in the same way. At least 30 percent of these patients will fail the first line of therapy, thus leading to further rounds of treatment and the increased probability of treatment failure, metastases, and ultimately, death. A superior approach to treatment would be the customization of therapy for the particular individual. The only current therapy that lends itself to customization is surgery. Chemotherapy and radiation treatment cannot be tailored to the patient, and surgery by itself, in most cases is inadequate for producing cures.

With the advent of monoclonal antibodies, the possibility of developing methods for customized therapy became more realistic since each antibody can be directed to a single epitope. Furthermore, it is possible to produce a combination of antibodies that are directed to the constellation of epitopes that uniquely define a particular individual's tumor.

Having recognized that a significant difference between cancerous and normal cells is that cancerous cells contain antigens that are specific to transformed cells, the scientific community has long held that monoclonal antibodies can be designed to specifically target transformed cells by binding specifically to these cancer antigens; thus giving rise to the belief that monoclonal antibodies can serve as “Magic Bullets” to eliminate cancer cells.

Monoclonal antibodies isolated in accordance with the teachings of the instantly disclosed invention have been shown to modify the cancerous disease process in a manner which is beneficial to the patient, for example by reducing the tumor burden, and will variously be referred to herein as cancerous disease modifying antibodies (CDMAB) or “anti-cancer” antibodies.

At the present time, the cancer patient usually has few options of treatment. The regimented approach to cancer therapy has produced improvements in global survival and morbidity rates. However, to the particular individual, these improved statistics do not necessarily correlate with an improvement in their personal situation.

Thus, if a methodology was put forth which enabled the practitioner to treat each tumor independently of other patients in the same cohort, this would permit the unique approach of tailoring therapy to just that one person. Such a course of therapy would, ideally, increase the rate of cures, and produce better outcomes, thereby satisfying a long-felt need.

Historically, the use of polyclonal antibodies has been used with limited success in the treatment of human cancers. Lymphomas and leukemias have been treated with human plasma, but there were few prolonged remissions or responses. Furthermore, there was a lack of reproducibility and no additional benefit compared to chemotherapy. Solid tumors such as breast cancers, melanomas and renal cell carcinomas have also been treated with human blood, chimpanzee serum, human plasma and horse serum with correspondingly unpredictable and ineffective results.

There have been many clinical trials of monoclonal antibodies for solid tumors. In the 1980s there were at least 4 clinical trials for human breast cancer which produced only 1 responder from at least 47 patients using antibodies against specific antigens or based on tissue selectivity. It was not until 1998 that there was a successful clinical trial using a humanized anti-Her2 antibody in combination with cisplatin. In this trial 37 patients were accessed for responses of which about a quarter had a partial response rate and another half had minor or stable disease progression.

The clinical trials investigating colorectal cancer involve antibodies against both glycoprotein and glycolipid targets. Antibodies such as 17-1A, which has some specificity for adenocarcinomas, had undergone Phase 2 clinical trials in over 60 patients with only 1 patient having a partial response. In other trials, use of 17-1A produced only 1 complete response and 2 minor responses among 52 patients in protocols using additional cyclophosphamide. Other trials involving 17-1A yielded results that were similar. The use of a humanized murine monoclonal antibody initially approved for imaging also did not produce tumor regression. To date there has not been an antibody that has been effective for colorectal cancer. Likewise there have been equally poor results for lung, brain, ovarian, pancreatic, prostate, and stomach cancers. There has been some limited success in the use of an anti-GD3 monoclonal antibody for melanoma. Thus, it can be seen that despite successful small animal studies that are a prerequisite for human clinical trials, the antibodies that have been tested thus far, have been for the most part, ineffective.

Prior Patents:

U.S. Pat. No. 5,750,102 discloses a process wherein cells from a patient's tumor are transfected with MHC genes which may be cloned from cells or tissue from the patient. These transfected cells are then used to vaccinate the patient.

U.S. Pat. No. 4,861,581 discloses a process comprising the steps of obtaining monoclonal antibodies that are specific to an internal cellular component of neoplastic and normal cells of the mammal but not to external components, labeling the monoclonal antibody, contacting the labeled antibody with tissue of a mammal that has received therapy to kill neoplastic cells, and determining the effectiveness of therapy by measuring the binding of the labeled antibody to the internal cellular component of the degenerating neoplastic cells. In preparing antibodies directed to human intracellular antigens, the patentee recognizes that malignant cells represent a convenient source of such antigens.

U.S. Pat. No. 5,171,665 provides a novel antibody and method for its production. Specifically, the patent teaches formation of a monoclonal antibody which has the property of binding strongly to a protein antigen associated with human tumors, e.g. those of the colon and lung, while binding to normal cells to a much lesser degree.

U.S. Pat. No. 5,484,596 provides a method of cancer therapy comprising surgically removing tumor tissue from a human cancer patient, treating the tumor tissue to obtain tumor cells, irradiating the tumor cells to be viable but non-tumorigenic, and using these cells to prepare a vaccine for the patient capable of inhibiting recurrence of the primary tumor while simultaneously inhibiting metastases. The patent teaches the development of monoclonal antibodies which are reactive with surface antigens of tumor cells. As set forth at col. 4, lines 45 et seq., the patentees utilize autochthonous tumor cells in the development of monoclonal antibodies expressing active specific immunotherapy in human neoplasia.

U.S. Pat. No. 5,693,763 teaches a glycoprotein antigen characteristic of human carcinomas is not dependent upon the epithelial tissue of origin.

U.S. Pat. No. 5,783,186 is drawn to anti-Her2 antibodies which induce apoptosis in Her2 expressing cells, hybridoma cell lines producing the antibodies, methods of treating cancer using the antibodies and pharmaceutical compositions including said antibodies.

U.S. Pat. No. 5,849,876 describes new hybridoma cell lines for the production of monoclonal antibodies to mucin antigens purified from tumor and non-tumor tissue sources.

U.S. Pat. No. 5,869,268 is drawn to a method for generating a human lymphocyte producing an antibody specific to a desired antigen, a method for producing a monoclonal antibody, as well as monoclonal antibodies produced by the method. The patent is particularly drawn to the production of an anti-HD human monoclonal antibody useful for the diagnosis and treatment of cancers.

U.S. Pat. No. 5,869,045 relates to antibodies, antibody fragments, antibody conjugates and single chain immunotoxins reactive with human carcinoma cells. The mechanism by which these antibodies function is two-fold, in that the molecules are reactive with cell membrane antigens present on the surface of human carcinomas, and further in that the antibodies have the ability to internalize within the carcinoma cells, subsequent to binding, making them especially useful for forming antibody-drug and antibody-toxin conjugates. In their unmodified form the antibodies also manifest cytotoxic properties at specific concentrations.

U.S. Pat. No. 5,780,033 discloses the use of autoantibodies for tumor therapy and prophylaxis. However, this antibody is an anti-nuclear autoantibody from an aged mammal. In this case, the autoantibody is said to be one type of natural antibody found in the immune system. Because the autoantibody comes from “an aged mammal”, there is no requirement that the autoantibody actually comes from the patient being treated. In addition the patent discloses natural and monoclonal anti-nuclear autoantibody from an aged mammal, and a hybridoma cell line producing a monoclonal anti-nuclear autoantibody.

### SUMMARY OF THE INVENTION

The instant inventors have previously been awarded U.S. Pat. No. 6,180,357, entitled “Individualized Patient Specific Anti-Cancer Antibodies” directed to a process for selecting individually customized anti-cancer antibodies which are useful in treating a cancerous disease. For the purpose of this document, the terms “antibody” and “monoclonal antibody” (mAb) may be used interchangeably and refer to intact immunoglobulins produced by hybridomas, immunoconjugates and, as appropriate, immunoglobulin fragments and recombinant proteins derived from immunoglobulins, such as chimeric and humanized immunoglobulins, F(ab′) and F(ab′)₂ fragments, single-chain antibodies, recombinant immunoglobulin variable regions (Fv)s etc. Furthermore, it is within the purview of this invention to conjugate standard chemotherapeutic modalities, e.g. radionuclides, with the CDMAB of the instant invention, thereby focusing the use of said chemotherapeutics. The CDMAB can also be conjugated to toxins, cytotoxic moieties or enzymes e.g. biotin conjugated enzymes.

The prospect of individualized anti-cancer treatment will bring about a change in the way a patient is managed. A likely clinical scenario is that a tumor sample is obtained at the time of presentation, and banked. From this sample, the tumor can be typed from a panel of pre-existing cancerous disease modifying antibodies. The patient will be conventionally staged but the available antibodies can be of use in further staging the patient. The patient can be treated immediately with the existing antibodies and/or a panel of antibodies specific to the tumor can be produced either using the methods outlined herein or through the use of phage display libraries in conjunction with the screening methods herein disclosed. All the antibodies generated will be added to the library of anti-cancer antibodies since there is a possibility that other tumors can bear some of the same epitopes as the one that is being treated. The antibodies produced according to this method may be useful to treat cancerous disease in any number of patients who have cancers that bind to these antibodies.

Using substantially the process of U.S. Pat. No. 6,180,370, the mouse monoclonal antibodies 7BD-33-11A and 1A245.6 were obtained following immunization of mice with cells from a patient's breast tumor biopsy. Within the context of this application, anti-cancer antibodies having either cell-killing (cytotoxic) or cell-growth inhibiting (cytostatic) properties will hereafter be referred to as cytotoxic. These antibodies can be used in aid of staging and diagnosis of a cancer, and can be used to treat tumor metastases. The 7BD-33-11A and 1A245.6 antigen was expressed on the cell surface of a broad range of human cell lines from different tissue origins. The breast cancer cell line MCF-7 and prostate cancer cell line PC-3 were the only 2 cancer cell lines tested that were susceptible to the cytotoxic effects of either 7BD-33-11A or 1A245.6.

The result of 7BD-33-11A and 1A245.6 cytotoxicity against breast and prostate cancer cells in culture was further extended by establishing its anti-tumor activity in vivo. In an in vivo model of human cancer, the MB-231 breast cancer cells or PC-3 prostate cancer cells were implanted underneath the skin at the scruff of the neck of severe combined immunodeficient (SCID) mice, as they are incapable of rejecting the human tumor cells due to a lack of certain immune cells. Pre-clinical xenograft tumor models are considered valid predictors of therapeutic efficacy. Xenografts in mice grow as solid tumors developing stroma, central necrosis and neo-vasculature in the same manner as naturally occurring cancers. The mammary tumor cell line MB-231 and the prostate tumor cell line PC-3 have been evaluated as an in vivo xenograft model in immunodeficient mice. The successful engraftment or ‘take-rate’ of both the MB-231 and PC-3 tumors and the sensitivity of the tumors to standard chemotherapeutic agents have characterized them as suitable models. The MB-231 parental cell line and variants of the cell line and the PC-3 androgen-independent cell line have been used successfully in xenograft tumor models to evaluate a wide range of therapeutic agents that used as clinical chemotherapeutic agents.

As outlined and described in Ser. No. 10/348,284, 7BD-33-11A and 1A245.6 prevented tumor growth and reduced tumor burden in a preventative in vivo model of human breast cancer. Monitoring continued past 150 days post-treatment. 7BD-33-11A never developed tumors and 87.5 percent of the 7BD-33-11A treatment group was still alive at over 6 months post-implantation. Conversely, the isotype control group had 100 percent mortality by day 72 (23 days post-treatment). 1A245.6 treated mice reached 100 percent mortality by day 151 post-treatment, which is greater than 6 times longer than the isotype control treatment group. Therefore 1A245.6, and to a greater extent 7BD-33-11A, enhanced survival and decreased the tumor burden in a breast cancer model.

Also as outlined and described in Ser. No. 10/348,284, both 7BD-33-11A and 1A245.6 significantly suppressed tumor growth and decreased tumor burden in an established in vivo model of human breast cancer. By day 80 (23 days post-treatment), 7BD-33-11A treated mice had 83 percent lower mean tumor volumes in comparison to isotype control group (p=0.001). 1A245.6 treatment also produced lower mean tumor volumes on this day, 35 percent (p=0.135). Using survival as a measure of antibody efficacy, it was estimated that the risk of dying in the 7BD-33-11A treatment group was about 16 percent of the isotype control group (p=0.0006) at around 60 days post-treatment. 100 percent of the isotype control group died by 50 days post-treatment. In comparison, 1A245.6 treated mice survived until 100 days post-treatment and 60% of the 7BD-33-11A treatment groups were still alive at 130 days post-treatment. This data demonstrate that both 1A245.6 and 7BD-33-11A treatments conferred a survival and reduced tumor burden benefit compared to the control treated group. 7BD-33-11A and 1A245.6 treatment appeared safe, as it did not induce any signs of toxicity, including reduced body weight and clinical distress. Thus, 7BD-33-11A and 1A245.6 treatment was efficacious as it both delayed tumor growth and enhanced survival compared to the control-treated groups in a well-established model of human breast cancer.

In addition to the beneficial effects in the established in vivo tumor model of breast cancer, 7BD-33-11A and 1A245.6 treatment also had anti-tumor activity against PC-3 cells in a preventative in vivo prostate cancer model. In this prostate xenograft model, 7BD-33-11A and 1A245.6 were given separately to mice 1 day prior to implantation of tumor cells followed by weekly injections for 7 weeks. 7BD-33-11A and 1A245.6 treatment was significantly (p=0.001 and 0.017 respectively) more effective in suppressing tumor growth shortly after the treatment period than an isotype control antibody. At the end of the treatment phase, mice given 7BD-33-11A or 1A245.6 had tumors that grew to only 31 and 50 percent of the isotype control group respectively.

For PC-3 SCID xenograft models, body weight can be used as a surrogate indicator of disease progression. On day 52, both 7BD-33-11A and 1A245.6 treatment significantly (p=0.002 and 0.004 respectively) prevented the loss of body weight by 54 and 25 percent respectively in comparison to isotype control. Mice were monitored for survival post-treatment. At 11 days post-treatment, isotype and buffer control mice had reached 100 percent mortality. Conversely, 7BD-33-11A and 1A245.6 reached 100 percent mortality at day 38 post-treatment, 3 times longer than the control groups. Thus, 7BD-33-11A and 1A245.6 treatment was efficacious as it both delayed tumor growth, prevented body weight loss and extended survival compared to the isotype control treated group in a well-established model of human prostate cancer.

In addition to the preventative in vivo tumor model of prostate cancer, 7BD-33-11A demonstrated anti-tumor activity against PC-3 cells in an established in vivo tumor model. In this xenograft model, PC-3 prostate cancer cells were transplanted subcutaneously into SCID mice such that the tumor reached a certain size before antibody treatment. Treatment with 7BD-33-11A was again compared to isotype control. It was shown that the 7BD-33-11A treatment group had significantly (p<0.024) smaller mean tumor volumes compared with the isotype control treated group immediately following treatment. 7BD-33-11A treatment mediated tumor suppression by 36 percent compared to the isotype control group. The anti-tumor activities of 7BD-33-11A, in several different cancer models, make it an attractive anti-cancer therapeutic agent.

The binding of 7BD-33-11A and 1A245.6 towards normal human tissues was determined. By IHC staining, the majority of the tissues failed to express the 7BD-33-11A antigen, including the vital organs, such as the kidney, heart, and lung. 7BD-33-11A stained the salivary gland, liver, pancreas, stomach, prostate and duodendum, and strongly stained the tonsil. Results from tissue staining indicated that 7BD-33-11A showed restricted binding to various cell types but had binding to infiltrating macrophages, lymphocytes, and fibroblasts. For 1A245.6, a wider range of tissues was positively stained. For the majority of cases, staining was restricted to the epithelium or infiltrating macrophages, lymphocytes, and fibroblasts. However, positive staining was seen on both cardiac muscle and hepatocytes. 7BD-33-11A and 1A245.6 displayed both membrane and cytoplasmic staining patterns.

Localization of the 7BD-33-11A and 1A245.6 antigen and its prevalence within breast cancer patients is important in assessing the benefits of immunotherapy to. To address antigen expression in breast tumors from cancer patients, tumor tissue samples from 50 individual breast cancer patients were screened for expression of either the 7BD-33-11A or 1A245.6 antigen. The results of the study showed that 36 percent of tissue samples positively stained for the 7BD-33-11A antigen. Expression of 7BD-33-11A within patient samples appeared specific for cancer cells as staining was restricted to malignant cells. In addition, 7BD-33-11A stained 0 of 10 samples of normal tissue from breast cancer patients. On the other hand, 1A245.6 stained 98 percent of breast cancer tissue samples. 1A245.6 also stained 8 out of 10 samples of normal tissue from breast cancer patients. However, in general this staining was much weaker than that observed with the breast cancer tissue samples and was generally restricted to infiltrating fibroblasts. 7BD-33-11A and 1A245.6 expression was further evaluated based on breast tumor expression of the receptors for the hormones estrogen and progesterone, which play an important role in the development, treatment, and prognosis of breast tumors. No correlation was apparent between expression of the 1A245.6 antigen and expression of the receptors for either estrogen or progesterone. There was a slight correlation between estrogen or progesterone receptor expression and expression of 7BD-33-11A; tissues with receptor expression had slightly higher 7BD-33-11A expression. When tumors were analyzed based on their stage, or degree to which the cancer advanced, results suggested a trend towards greater positive expression with higher tumor stage for 7BD-33-11A and higher intensity staining with higher tumor stage for 1A245.6. However, the results were limited by the small sample size.

To further extend the potential therapeutic benefit of 7BD-33-11A and 1A245.6, the frequency and localization of the antigen within various human cancer tissues was determined. Several cancer types, in addition to breast cancer, expressed the 7BD-33-11A antigen. The positive human cancer types included skin (1/2), lung (3/4), liver (2/3), stomach (4/5), thyroid (2/2), prostate (1/1), uterus (4/4) and kidney (3/3). Some cancers did not express the antigen; these included ovary (0/3), testis (0/1), brain (0/2) and lymph node (0/2). For 1A245.6, as with the normal human tissue array, a multitude of cancers from various human tissue types were positively stained. Greater staining was seen on malignant cells of the skin, lung, liver, uterus, kidney, stomach and bladder. As with human breast cancer tissue, localization of 7BD-33-11A and 1A245.6 occurred both on the membrane and within the cytoplasm of these tumor cells. So, in addition to the 7BD-33-11A and 1A245.6 antibody binding to cancer cell lines in vitro, there is evidence that the antigen is expressed in humans, and on multiple types of cancers.

In toto, this data demonstrates that both the 7BD-33-11A and 1A245.6 antigen is a cancer associated antigen and is expressed in humans, and is a pathologically relevant cancer target. Further, this data also demonstrates the binding of 7BD-33-11A and 1A245.6 antibody to human cancer tissues, and can be used appropriately for assays that can be diagnostic, predictive of therapy, or prognostic. In addition, the cell membrane localization of this antigen permits the use of this antigen, its gene or derivatives, its protein or its variants to be used for assays that can be diagnostic, predictive of therapy, or prognostic.

In all, this invention teaches the use of the 7BD-33-11A or 1A245.6 antigen as a target for a therapeutic agent, that when administered can reduce the tumor burden of a cancer expressing the antigen in a mammal, and can also lead to a prolonged survival of the treated mammal. This invention also teaches the use of CDMAB (7BD-33-11A/1A245.6), and its derivatives, to target its antigen to reduce the tumor burden of a cancer expressing the antigen in a mammal, and to prolong the survival of a mammal bearing tumors that express this antigen. Furthermore, this invention also teaches the use of detecting the 7BD-33-11A or 1A245.6 antigen in cancerous cells that can be useful for the diagnosis, prediction of therapy, and prognosis of mammals bearing tumors that express this antigen.

If a patient is refractory to the initial course of therapy or metastases develop, the process of generating specific antibodies to the tumor can be repeated for re-treatment. Furthermore, the anti-cancer antibodies can be conjugated to red blood cells obtained from that patient and re-infused for treatment of metastases. There have been few effective treatments for metastatic cancer and metastases usually portend a poor outcome resulting in death. However, metastatic cancers are usually well vascularized and the delivery of anti-cancer antibodies by red blood cells can have the effect of concentrating the antibodies at the site of the tumor. Even prior to metastases, most cancer cells are dependent on the host's blood supply for their survival and an anti-cancer antibody conjugated to red blood cells can be effective against in situ tumors as well. Alternatively, the antibodies may be conjugated to other hematogenous cells, e.g. lymphocytes, macrophages, monocytes, natural killer cells, etc.

There are five classes of antibodies and each is associated with a function that is conferred by its heavy chain. It is generally thought that cancer cell killing by naked antibodies are mediated either through antibody-dependent cell-mediated cytotoxicity (ADCC) or complement-dependent cytotoxicity (CDC). For example murine IgM and IgG2a antibodies can activate human complement by binding the C-1 component of the complement system thereby activating the classical pathway of complement activation which can lead to tumor lysis. For human antibodies, the most effective complement activating antibodies are generally IgM and IgG1. Murine antibodies of the IgG2a and IgG3 isotype are effective at recruiting cytotoxic cells that have Fc receptors which will lead to cell killing by monocytes, macrophages, granulocytes and certain lymphocytes. Human antibodies of both the IgG1 and IgG3 isotype mediate ADCC.

Another possible mechanism of antibody mediated cancer killing may be through the use of antibodies that function to catalyze the hydrolysis of various chemical bonds in the cell membrane and its associated glycoproteins or glycolipids, so-called catalytic antibodies.

There are two additional mechanisms of antibody mediated cancer cell killing which are more widely accepted. The first is the use of antibodies as a vaccine to induce the body to produce an immune response against the putative antigen that resides on the cancer cell. The second is the use of antibodies to target growth receptors and interfere with their function or to down regulate that receptor so that effectively its function is lost.

Accordingly, it is an objective of the invention to utilize a method for producing CDMAB from cells derived from a particular individual which are cytotoxic with respect to cancer cells while simultaneously being relatively non-toxic to non-cancerous cells, in order to isolate hybridoma cell lines and the corresponding isolated monoclonal antibodies and antigen binding fragments thereof for which said hybridoma cell lines are encoded.

It is an additional objective of the invention to teach CDMAB and antigen binding fragments thereof.

It is a further objective of the instant invention to produce CDMAB whose cytotoxicity is mediated through ADCC.

It is yet an additional objective of the instant invention to produce CDMAB whose cytotoxicity is mediated through CDC.

It is still a further objective of the instant invention to produce CDMAB whose cytotoxicity is a function of their ability to catalyze hydrolysis of cellular chemical bonds.

A still further objective of the instant invention is to produce CDMAB which are useful in a binding assay for the diagnosis, prognosis, and monitoring of cancer.

Other objects and advantages of this invention will become apparent from the following description wherein are set forth, by way of illustration and example, certain embodiments of this invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Survival of tumor-bearing mice after treatment with 7BD-33-11A, 1A245.6 or isotype control antibody in a preventative MB-231 xenograft study. Mice were monitored for survival for 200 days post-treatment.

FIG. 2. Effect of 7BD-33-11A and 1A245.6 on tumor growth in a preventative MB-231 breast cancer model. The dashed line indicates the period during which the antibody was administered. Data points represent the mean +/− SEM.

FIG. 3. Survival of tumor-bearing mice after treatment with 7BD-33-11A, 1A245.6 or isotype control antibody in an established MB-231 xenograft study. Mice were monitored for survival for 130 days post-treatment.

FIG. 4. Effect of 7BD-33-11A and 1A245.6 on tumor growth in a preventative PC-3 prostate cancer model. The dashed line indicates the period during which the antibody was administered. Data points represent the mean +/− SEM.

FIG. 5. Histogram showing mean body weight of the different treatment groups over the duration of the preventative PC-3 xenograft study. Data are presented as the mean +/− SEM for each group at each time point.

FIG. 6. Survival of tumor-bearing mice after treatment with 7BD-33-11A, 1A245.6, isotype or buffer control antibody in a preventative PC-3 xenograft study. Mice were monitored for survival for 38 days post-treatment.

FIG. 7. Effect of 7BD-33-11A and 1A245.6 on tumor growth in an established PC-3 prostate cancer model. The dashed line indicates the period during which the antibody was administered. Data points represent the mean +/− SEM.

FIG. 8. Histogram showing mean body weight of the different treatment groups over the duration of the established PC-3 xenograft study. Data are presented as the mean +/− SEM for each group at each time point.

FIG. 9. Normal Human Brain A. 7BD-33-11A. B. 1A245.6. C. Negative isotype control. Magnification is 200×.

FIG. 10. Normal Human Heart A. 7BD-33-11A. B. 1A245.6 (arrows indicate positive staining). C. Negative isotype control. Magnification is 200×.

FIG. 11. Normal Human Stomach Antrum. A. 7BD-33-11A (arrows indicate positive staining of gastric gland epithelium). B. 1A245.6 (arrows indicate positive staining of gastric gland epithelium). C. Negative isotype control. Magnification is 200×.

FIG. 12. Representative micrograph of 7BD-33-11A binding to human breast cancer tumor (infiltrating duct carcinoma; Panel A; black arrows: sheets of tumor cells, yellow arrow: tumor stroma) and human normal breast (Panel B). Magnification is 200×.

FIG. 13. Representative micrograph of 1A245.6 binding to human breast cancer tumor (infiltrating duct carcinoma; Panel A; black arrows: sheets of tumor cells, yellow arrow: tumor stroma) and human normal breast (Panel B; black arrows: fibroblasts). Magnification is 200×.

FIG. 14. Renal Cell Carcinoma. A. 7BD-33-11A (arrows indicate positive staining in sheets of tumor cells). B. 1A245.6 (arrows indicate positive staining in sheets of tumor cells). C. Negative isotype control. Magnification is 200×.

### DETAILED DESCRIPTION OF THE INVENTION

### EXAMPLE 1

The hybridoma cell lines 7BD-33-11A and 1A245.6 were deposited, in accordance with the Budapest Treaty, with the American Type Culture Collection, 10801 University Blvd., Manassas, Va. 20110-2209 on Jan. 8, 2003, under Accession Number PTA-4890 and PTA-4889 respectively. In accordance with 37 CFR 1.808, the depositors assure that all restrictions imposed on the availability to the public of the deposited materials will be irrevocably removed upon the granting of a patent.

7BD-33-11A and 1A245.6 monoclonal antibody was produced by culturing the hybridomas in CL-1000 flasks (BD Biosciences, Oakville, ON) with collections and reseeding occurring twice/week and with purification according to standard antibody purification procedures with Protein G Sepharose 4 Fast Flow (Amersham Biosciences, Baie d'Urfé, QC).

In Vivo MB-231 Preventative Survival Tumor Experiments

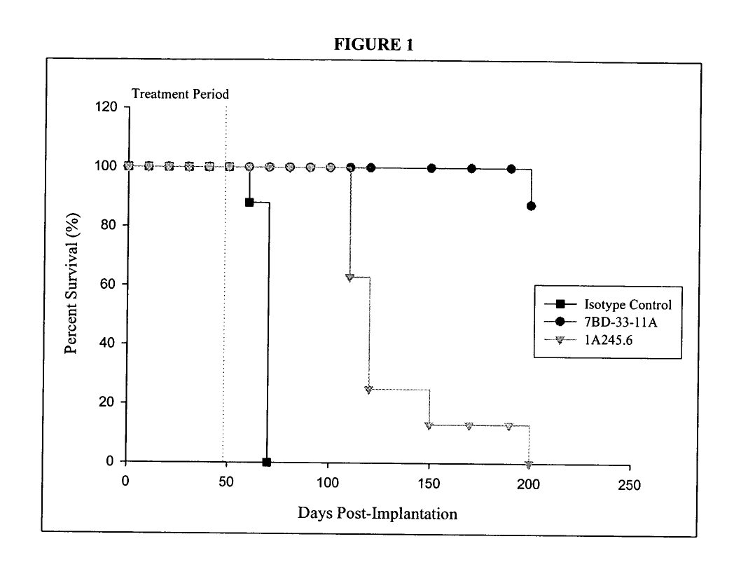

As outlined in Ser. No. 10/348,284, and with reference to FIG. 1, 4 to 8 week old female SCID mice were implanted with 5 million MB-231 human breast cancer cells in 100 microlitres saline injected subcutaneously in the scruff of the neck. The mice were randomly divided into 3 treatment groups of 10. On the day prior to implantation, 20 mg/kg of either 7BD-33-11A, 1A245.6 test antibodies or isotype control antibody (known not to bind MB-231 or PC-3 cells) was administered intraperitoneally at a volume of 300 microliters after dilution from the stock concentration with a diluent that contained 2.7 mM KCl, 1 mM KH₂PO₄, 137 mM NaCl and 20 mM Na₂HPO₄. The antibodies were then administered once per week for a period of 7 weeks in the same fashion. Tumor growth was measured about every seventh day with calipers for up to 10 weeks or until individual animals reached the Canadian Council for Animal Care (CCAC) end-points. Body weights of the animals were recorded for the duration of the study. At the end of the study all animals were euthanised according to CCAC guidelines.

In continuation from Ser. No. 10/348,284, there was a post-treatment survival benefit (FIG. 1) associated with treatment with either 7BD-33-11A or 1A245.6. 7BD-33-11A never developed tumors and only 1 mouse had died by day 200 (151 days post-treatment). In contrast, all of the isotype control mice had died by day 23 post-treatment. The 1A245.6 treated group did not reach 100 percent mortality until day 151 post-treatment which is greater than 6 times longer than the isotype control treatment group. In summary 1A245.6 and 7BD-33-11A increased survival and decreased tumor burden in a breast tumor model of human cancer.

### EXAMPLE 2

In Vivo MB-231 Established Tumor Experiments

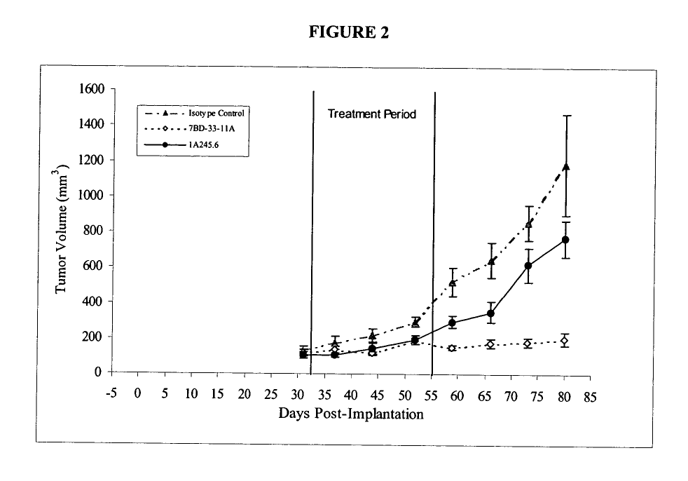

As outlined in Ser. No. 10/348,284, and with reference to FIGS. 2 and 3, 5 to 6 week old female SCID mice were implanted with 5 million MB-231 human breast cancer cells in 100 microlitres saline injected subcutaneously in the scruff of the neck. Tumor growth was measured with calipers every week. When the majority of the cohort reached a tumor volume of 100 mm³ (range 50-200 mm³) at 34 days post-implantation 8-10 mice were randomly assigned into each of 3 treatment groups. 7BD-33-11A, 1A245.6 test antibodies or isotype control antibody was administered intraperitoneally with 15 mg/kg of antibodies at a volume of 150 microliters after dilution from the stock concentration with a diluent that contained 2.7 mM KCl, 1 mM KH₂PO₄, 137 mM NaCl and 20 mM Na₂HPO₄. The antibodies were then administered 3 times per week for 10 doses in total in the same fashion until day 56 post-implantation. Tumor growth was measured about every seventh day with calipers until day 59 post-implantation or until individual animals reached the CCAC end-points. Body weights of the animals were recorded for the duration of the study. At the end of the study all animals were euthanised according to CCAC guidelines.

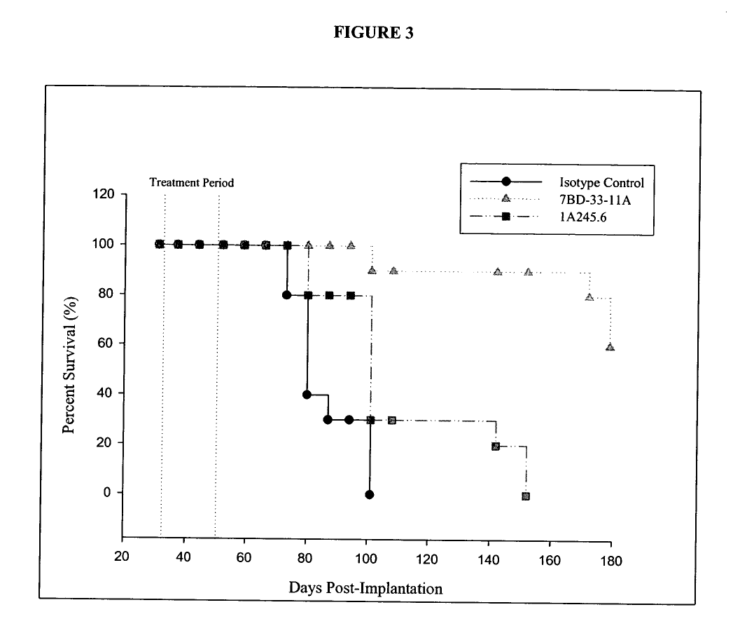

In continuation from Ser. No. 10/348,284, there was a post-treatment tumor burden reduction (FIG. 2) and survival benefit (FIG. 3) associated with treatment with either 7BD-33-11A or 1A245.6. At day 80 (23 days post-treatment) both 7BD-33-11A and 1A245.6 had decreased mean tumor volumes compared to isotype control treatment; 83 (p=0.001) and 35 percent (p=0.135) respectively. A Cox proportional model was used to compare the hazard (risk) rates in the different groups. In this method, the hazard rate of every group is compared with the hazard of the isotype control group. At approximately 60 days post-treatment, the risk of dying in the 7BD-33-11A group was 16 percent in comparison to the isotype control treatment group (p=0.0006). The survival benefit associated with 7BD-33-11A appeared to continue on well past the 100 day post-treatment mark. At day 130 post-treatment, 7BD-33-11A had 60% survival while all of the isotype control mice had died at day 50 post-treatment. 1A245.6 had double the survival time in comparison to the isotype control: 100 versus 50 days post-treatment. Therefore both 7BD-33-11A and 1A245.6 lowered the tumor burden and increased survival in comparison to a control antibody in a well recognized model of human breast cancer disease suggesting pharmacologic and pharmaceutical benefits of these antibodies for therapy in other mammals, including man.

### EXAMPLE 3

In Vivo PC-3 Preventative Tumor Experiments

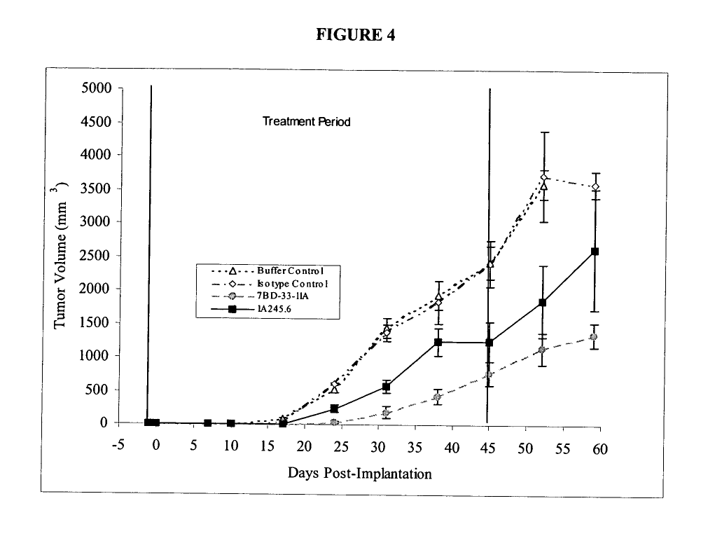

With reference to the data shown in FIGS. 4 and 5, 4 to 8 week old, male SCID mice were implanted with 1 million PC-3 human prostate cancer cells in 100 microliters saline injected subcutaneously in the scruff of the neck. The mice were randomly divided into 4 treatment groups of 8. On the day prior to implantation 20 mg/kg of 7BD-33-11A or 1A245.6 test antibody or isotype control antibody or buffer control was administered intraperitoneally at a volume of 300 microliters after dilution from the stock concentration with a diluent that contained 2.7 mM KCl, 1 mM KH₂PO₄, 137 mM NaCl and 20 mM Na₂HPO₄. The antibodies and buffer control were then administered once per week for a period of 7 weeks in the same fashion. Tumor growth was measured about every 7th day with calipers for up to 10 weeks or until individual animals reached the CCAC end-points or day 52. Body weights of the animals were recorded for the duration of the study. At the end of the study all animals were euthanised according to CCAC guidelines.

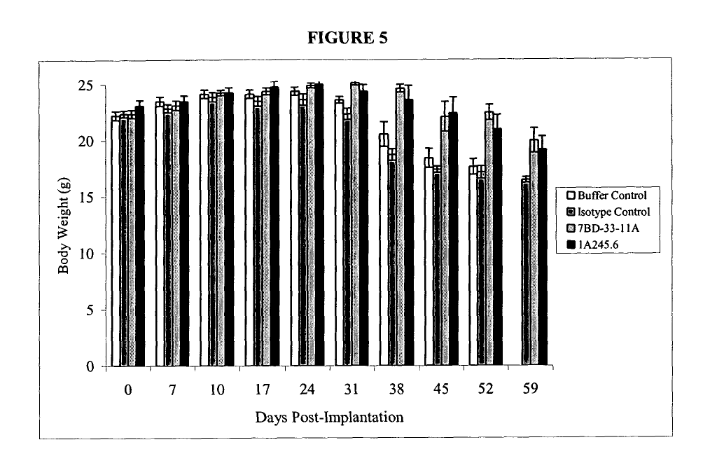

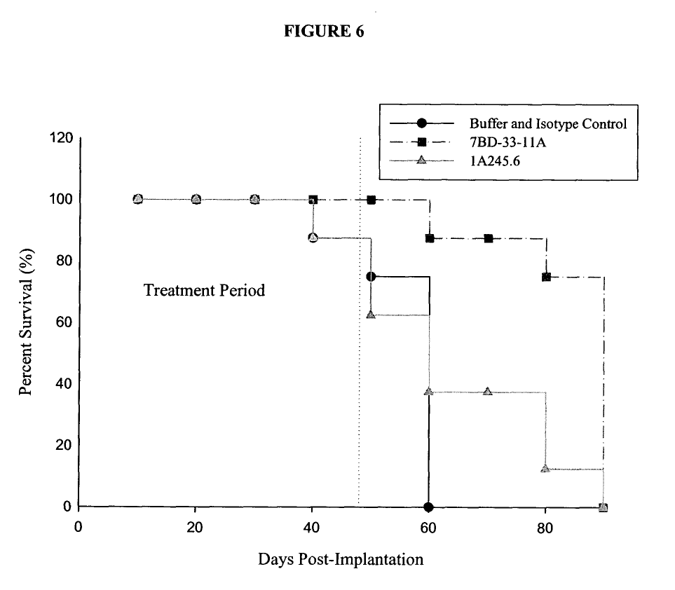

Using the least significant difference method (LSD) to specify the different groups, it is apparent that both 7BD-33-11A and 1A245.6 significantly reduced the tumor burden in treated mice in comparison to controls (FIG. 4). After treatment (day 52), 7BD-33-11A prevented tumor growth by 69 percent (p=0.001) in comparison to isotype control and 1A245.6 also prevented tumor growth by 50 percent (p=0.017) in comparison to isotype control. Similar findings were found when comparisons were made to the buffer control. In a PC-3 prostate cancer xenograft model, body weight can be used as a surrogate indicator of disease progression (FIG. 5). A repeated analysis of variance (Rep. ANOVA) indicates there was no significant difference in body weight between the isotype and buffer control group. Analysis of variance determined that at day 52, 7BD-33-11A had a significantly higher body weight than both of the control groups and the 1A245.6 treated group (p<0.03). Overall, 7BD-33-11A prevented body weight loss by 54 percent (p=0.002) while 1A245.6 prevented body weight loss by 25 percent (p=0.004) compared to the isotype control group. Mice were monitored post-treatment for survival (FIG. 6). 100 percent of 7BD-33-11A and 1A245.6 treated mice reached mortality by day 38 post-treatment, which is greater than 3 times longer than the isotype and buffer control treatment group, 11 days post-treatment.

In summary, 7BD-33-11A and 1A245.6 antibody treatment reduced tumor burden, delayed disease progression and extended survival in comparison to an isotype control antibody and a buffer control in a well-recognized model of human prostate cancer. These results suggest a potential pharmacologic and pharmaceutical benefit of these antibodies (7BD-33-11A and 1A245.6) as a therapy beyond breast cancer.

### EXAMPLE 4

In Vivo PC-3 Established Tumor Experiments:

Male SCID mice, 4 to 8 weeks old, were implanted with 1 million PC-3 prostate cancer cells in 100 microliters saline injected subcutaneously in the scruff of the neck. Tumor growth was measured with calipers every week. When the majority of the cohort reached a tumor volume of 275 mm³ (range 144-406 mm³) at 21 days post implantation, 9-10 mice were randomized into each of 4 treatment groups. 7BD-33-11A or 1A245.6 or isotype control antibody was administered intraperitoneally with 20 mg/kg/dose at a volume of 300 microliters after dilution from the stock concentration with a diluent that contained 2.7 mM KCl, 1 mM KH₂PO₄, 137 mM NaCl and 20 mM Na₂HPO₄. The antibodies were then administered 3 times per week for a total of 10 doses in the same fashion until day 43 post-implantation. Tumor growth was measured about every seventh day with calipers for the duration of the study or until individual animals reached CCAC end-points. Body weights of the animals were recorded for the duration of the study. At the end of the study all animals were euthanised according to CCAC guidelines.

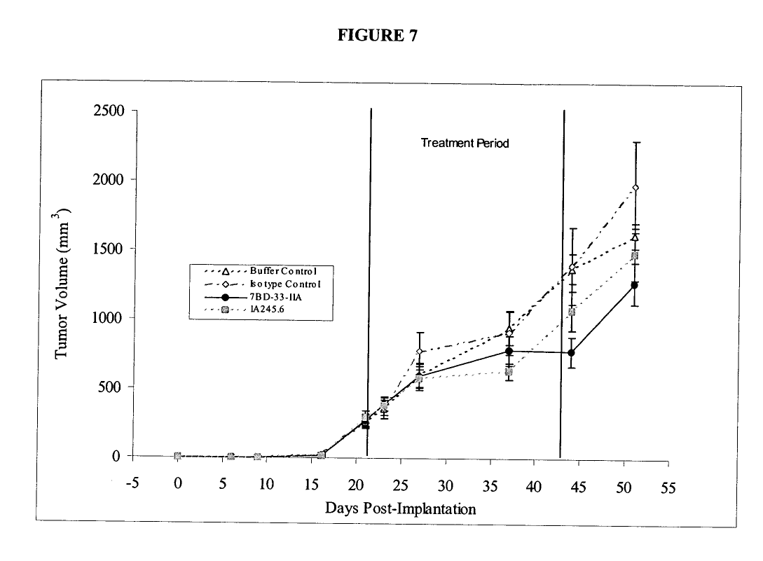

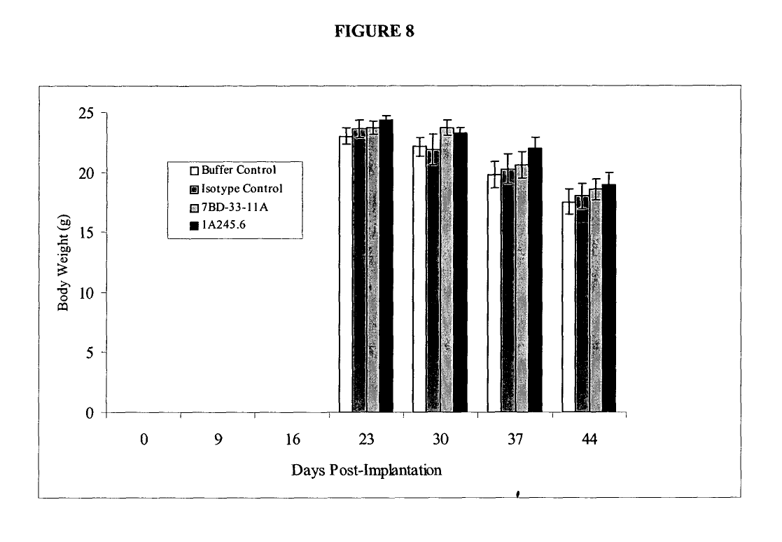

At the time of randomization the mean tumor volumes and the standard deviations in each group were similar. Statistically there was no difference in body weight between the groups. This indicated that true randomization had occurred. As shown in FIG. 7, the antibody 7BD-33-11A was able to significantly suppress tumor growth by 36 percent (pp=0.024) in comparison to isotype control at the end of the 3-week treatment period. 1A245.6 showed no significant difference when compared to isotype or buffer control treatment groups. Likewise, neither 7BD-33-11A or 1A245.6 showed any significant difference in comparison to isotype or buffer control treatment groups in terms of body weight (FIG. 8). All groups displayed the same significant amount of body weight loss throughout the study (p<0.001).

In summary, 7BD-33-11A is significantly more effective than the isotype control antibody in suppressing tumor growth in an established tumor xenograft model of prostate cancer in SCID mice. Therefore treatment with 7BD-33-11A significantly decreased the tumor burden of established tumors in two well-recognized models of human cancer disease (breast and prostate) suggesting pharmacologic and pharmaceutical benefits of this antibody for therapy in other mammals, including man.

### EXAMPLE 5

Normal Human Tissue Staining

IHC studies were conducted to characterize the 7BD-33-11A and 1A245.6 antigen distribution in humans. IHC optimization studies were performed previously in order to determine the conditions for further experiments. 7BD-33-11A and 1A245.6 monoclonal antibody was produced and purified as stated above.

Tissue sections were deparaffinized by drying in an oven at 58° C. for 1 hour and dewaxed by immersing in xylene 5 times for 4 minutes each in Coplin jars. Following treatment through a series of graded ethanol washes (100%-75%) the sections were re-hydrated in water. The slides were immersed in 10 mM citrate buffer at pH 6 (Dako, Toronto, Ontario) then microwaved at high, medium, and low power settings for 5 minutes each and finally immersed in cold PBS. Slides were then immersed in 3% hydrogen peroxide solution for 6 minutes, washed with PBS three times for 5 minutes each, dried, incubated with Universal blocking solution (Dako, Toronto, Ontario) for 5 minutes at room temperature. 7BD-33-11A, 1A245.6, monoclonal mouse anti-vimentin (Dako, Toronto, Ontario) or isotype control antibody (directed towards Aspergillus niger glucose oxidase, an enzyme which is neither present nor inducible in mammalian tissues; Dako, Toronto, Ontario) were diluted in antibody dilution buffer (Dako, Toronto, Ontario) to its working concentration (5 μg/mL for each antibody) and incubated overnight for 1 hour at room temperature. The slides were washed with PBS 3 times for 5 minutes each. Immunoreactivity of the primary antibodies was detected/visualized with HRP conjugated secondary antibodies as supplied (Dako Envision System, Toronto, Ontario) for 30 minutes at room temperature. Following this step the slides were washed with PBS 3 times for 5 minutes each and a color reaction developed by adding DAB (3,3′-diaminobenzidine tetrahydrachloride, Dako, Toronto, Ontario) chromogen substrate solution for immunoperoxidase staining for 10 minutes at room temperature. Washing the slides in tap water terminated the chromogenic reaction. Following counterstaining with Meyer's Hematoxylin (Sigma Diagnostics, Oakville, ON), the slides were dehyrdated with graded ethanols (75-100%) and cleared with xylene. Using mounting media (Dako Faramount, Toronto, Ontario) the slides were coverslipped. Slides were microscopically examined using an Axiovert 200 (Zeiss Canada, Toronto, ON) and digital images acquired and stored using Northern Eclipse Imaging Software (Mississauga, ON). Results were read, scored and interpreted by a pathologist.

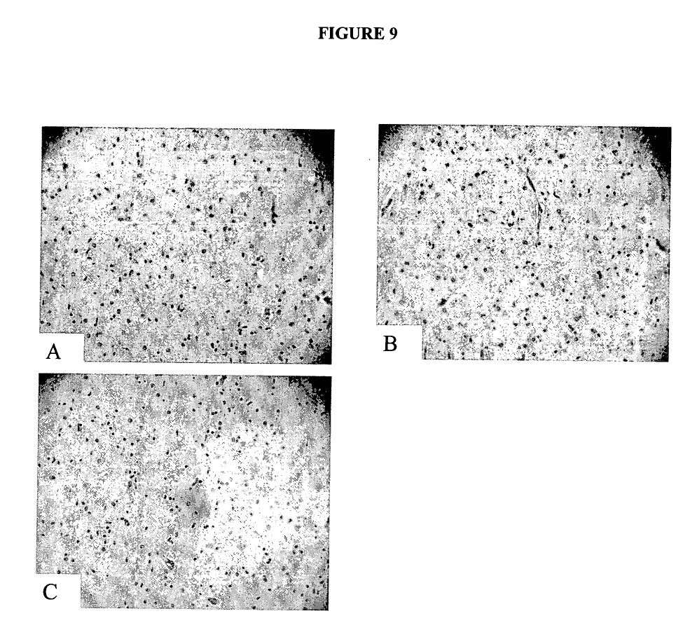

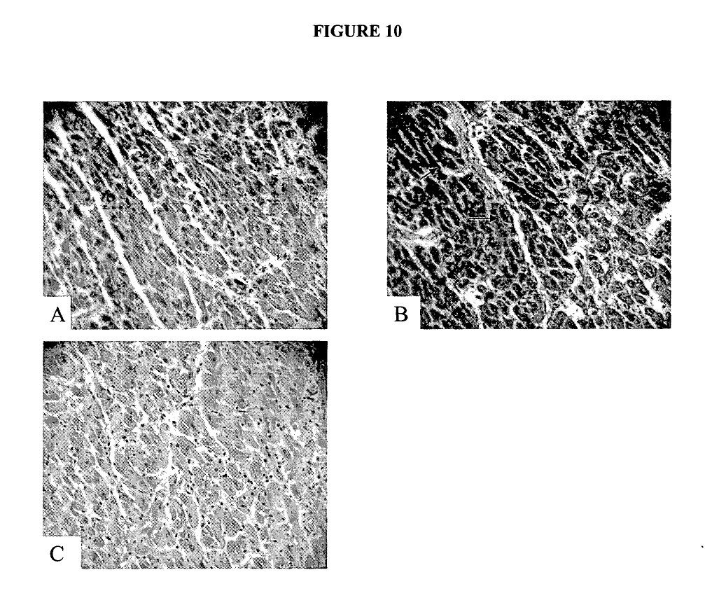

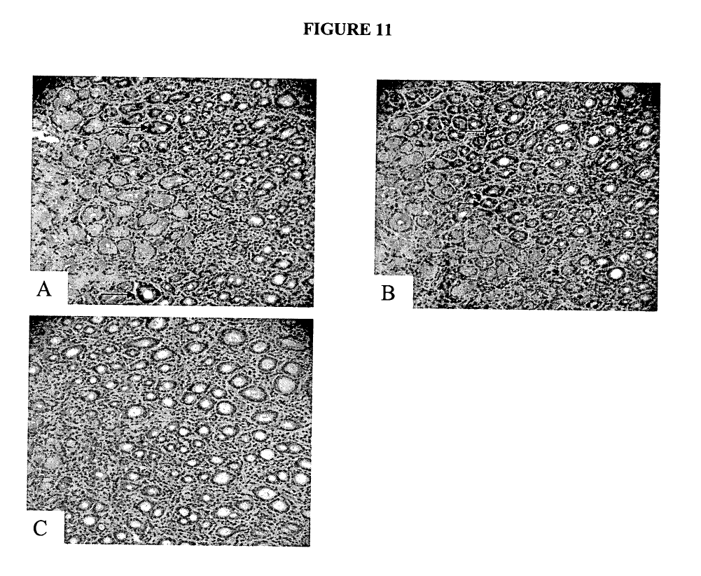

Binding of antibodies to 59 normal human tissues was performed using a human, normal organ tissue array (Imgenex, San Diego, Calif.). Table 1 presents a summary of the results of 7BD-33-11A and 1A245.6 staining of an array of normal human tissues. From the table, there are 3 categories of tissue staining. A group of tissues was completely negative. These tissues included normal skin, brain (FIG. 9A), ovary, thymus, thyroid, small bowel, esophaguas, heart (FIG. 10A), gall bladder and lymph node for 7BD-33-11A. For 1A245.6, the completely negative tissues comprised of skin, sub-cutis fat, esophagus and brain (FIG. 9B). A second group of tissues comprised tissues that demonstrated positive staining. These included the liver and pancreas for 7BD-33-11A. The tonsil had the strongest staining with this antibody. For 1A245.6, positive staining occurred in the liver, heart (FIG. 10B), testis, thyroid, adrenal gland and myometrium. As with 7BD-33-11A, 1A245.6 stained the tonsil the strongest. A third group of tissues included tissues in which staining was positive in the tissue section, but was limited to infiltrating macrophages, lymphocytes, fibroblasts or the epithelium, for example the stomach for both 7BD-33-11A and 1A245.6 (FIGS. 11A and B respectively). It should be noted that the 7BD-33-11A antigen is not present on cells of several of the vital organs, including kidney, heart (FIG. 10A) and lung. Overall, 7BD-33-11A binds to a smaller subset of normal human tissues compared to 1A245.6 with weak to moderate binding in the tissues that are positive. 1A245.6 staining, albeit more extensive, is also generally weak to moderate in intensity and in the majority of cases is limited to the epithelium of the stained tissue. These results suggest that the antigen for 7BD-33-11A is not widely expressed on normal tissues, and that the antibody would bind specifically to a limited number of tissues in humans. In addition, the antigen for 1A245.6, besides being present in the heart and liver, is limited to epithelium and infiltrating lymphocytes, macrophages and fibroblasts.

TABLE 1
IHC On Normal Human Tissue

| Sec No. | Organ | IA245.6 | 7BD-33-11A | Vimentin |
|---|---|---|---|---|
| 1 | *Skin | − | − | +++ Fibroblasts |
| 2 | *Skin | − | − | +++ Fibroblasts |
| 3 | Sub-cutis fat | − | − | ++ Adipocytes |
| 4 | Breast | +/− Fibroblasts | − | ++ Endothelium, Smooth muscles of blood |
| | | | | vessels |
| 5 | Breast | +++ Lobular epithelium | +/− Fibroblasts | ++ Blood vessels, stroma |
| 6 | Spleen | ++ Lymphocytes | +/− Lymphocytes | +++ [symbol] endothelium, Lymphocytes |
| 7 | Spleen | +++ Lymphocytes | +/− Lymphocytes | +++ [symbol] endothelium, Lymphocytes |
| 8 | Lymphnode | ++ Endothelium of blood vessels, Lymphocytes | − | +++ Lymphocytes |
| 9 | Lymphnode | + Endothelium of blood vessels | − | ++ Blood vessels, Lymphocytes |
| 10 | Skeletal muscle | +/− Endothelium of blood vessels | − | +/− Blood vessels |
| 11 | Nasal mucosa | − NR | − NR | − NR |
| 12 | Lung | +/− Interstitial cells (macrophages) | − | ++ Alevolar epithelium, Macrophages |
| 13 | Lung | +/− Bronchiolar epithelium, ++ Macrophages | + Macrophages | ++ Alevolar epithelium, Macrophages, |
| | | | | Lymphocytes |
| 14 | Bronchis | − NR | − NR | ++ Chondrocytes, NR |
| 15 | Heart | ++ Cardiac muscle | **− | +++ Blood vessels |
| 16 | Salivary gland | ++ Acinar epithelium | + Acinar epithelium | +++ Blood vessels, Peripheral nerves |
| 17 | Liver | +++ Hepatocytes | ++ Hepatocytes | ++ Blood vessels |
| 18 | Liver | +++ Hepatocytes | + Hepatocytes | *+++ Blood vessels, Macrophages |
| 19 | Liver | + Hepatocytes | +/− Hepatocytes | +/− Blood vessels |
| 20 | Gall bladder | ++ Mucosal epithelium, Fibroblasts, Smooth | − | +++ Lymphocytes |
| | | muscle fibers | | |
| 21 | Pancreas | +++ Acinar epithelium, Islets of Langherhans | + Acinar epithelium | +++ Acinar epithelium, Blood vessels |
| 22 | Pancreas | +++ Acinar epithelium, Islets of Langherhans | ++ Acinar epithelium, Islets | ++ Acinar epithelium, Blood vessels |
| | | | of Langherhans | |
| 23 | [symbol] | +++ Keratin, + Lymphocytes | +++ Keratin, +/− | +++ Lymphocytes |
| | | | Lymphocytes | |
| 24 | Esophagus | − | − | ++ Blood vessels |
| 25 | Esophagus | − | − | +++ Blood vessels |
| 26 | ***Stomach | +/− Gastric gland epithelium, ++ Lymphocytes | ++ Gastric gland epithelium | +++ Blood vessels & fibroblasts |
| | body | in lamina propria | | |
| 27 | ***Stomach | +++ Gastric gland epithelium, + Lymphocytes | ++ Gastric gland epithelium | +++ Lymphocytes, Blood vessels, |
| | body | in lamina propria | | Fibroblasts |
| 28 | Stomach | +/− Gastric gland epithelium, + Lymphocytes | ++ Gastric gland epithelium | +++ Lymphocytes |
| | antrum | | | |
| 29 | Stomach, | − | − | +++ Lymphocytes, Blood vessels |
| | Smooth muscle | | | |
| 30 | Duodenum | +++ Lymphocytes | + Intestinal gland epithelium | ++ Fibroblasts, Blood vessels |
| 31 | Small bowel | +/− Lymphocytes in lamina propria | − | + Lymphocytes, Blood vessels |
| 32 | Small bowel | +/− Lymphocytes in lamina propria | − | +++ Lymphocytes in lamina propria |
| 33 | Appendix | +++ Mucosal epithelium, Lymphocytes | ++ Lymphocytes | +++ Lymphocytes, Blood vessels |
| 34 | Colon | +/− Lymphocytes | +/− Macrophages in lamina | ++ Lymphocytes |
| | | | propria | |
| 35 | Colon | + Lymphocytes | − | ++ Lymphocytes, Blood vessels |
| 36 | Rectum | +/− Lymphocytes, Blood vessels | − | ++ Lymphocytes, Blood vessels |
| 37 | Kidney cortex | ++ Tubular epithelium | − | +++ Glomerular capillary, Blood vessels |
| 38 | Kidney cortex | +++ Tubular epithelium | + Tubular epithelium | +++ Tubular epithelium glomerular |
| | | | | capillary, Blood vessels |
| 39 | Kidney | ++ Tubular epithelium | − | ++ Renal tubule epithelium, Lipocytes, |
| | medulla | | | Fibroblasts |
| 40 | Urinary bladder | ++ Transitional epithelium | +/− Transitional epithelium | ++ Blood vessels |
| 41 | Prostate | +++ Glandular epithelium | ++ Glandular epithelium | +++ Glandular epithelium, Blood vessels |
| 42 | Prostate | +++ Glandular epithelium | ++ Glandular epithelium | +++ Glandular epithelium, Blood vessels |
| 43 | Seminal vesicle | + Mucosal epithelium | +/− Mucosal epithelium | +/− |
| 44 | Testis | ++ Germinal epithelium, + Leydig cells | +/− Leydig cells | +++ Germinal epithelium |
| 45 | Endometrium | +++ Glandular epithelium, + Stroma | − | +++ Endometrial glands, Stroma |
| | profilarative | | | |
| 46 | Endometrium | ++ Glandular epithelium, Stroma | − | ++ Glandular epithelium, +++ Blood vessels |
| | secretory | | | |
| 47 | Myometrium | + Smooth muscle fibers | +/− Fibroblasts | ++ Smooth muscle fibers, Blood vessels |
| 48 | Uterine cervix | +/− Fibroblasts | − | +++ Fibroblasts |
| 49 | Salpinx | + Mucosal epithelium, Blood vessels | − | +/− Mucosal epithelium, +++ Blood vessels |
| 50 | ****Ovary | + Stromal cells | − | +++ Stromal cells |
| 51 | Placenta villi | + Trophoblasts | − | ++ Blood vessels |
| 52 | Placenta villi | ++ Trophoblasts | − | ++ Blood vessels |
| 53 | Umbilical cord | − | − | ++ Fibroblasts |
| 54 | Adrenal gland | ++ Endocrine cells | **+/− | **+/− |
| 55 | Thyroid | +/− Follicular cells | − | ++ Follicular cells, Blood vessels |
| 56 | Thymus | + Lymphocytes | − | +++ Lymphocytes |
| 57 | Brain white | − | − | ++ Astrocytes |
| | matter | | | |
| 58 | Brain gray | − | − | ++ Blood vessels |
| | matter | | | |
| 59 | Cerebellum | − | − | ++ Cerebellar cortex |

Abbreviations:
*Originally pigmented stratum basale;
**Endogenous cytoplasmic pigment/back ground staining;
***Stomach antrum (not stomach body);
****Ovarian stroma only;
NR: The section is not representative;
CS: The section is completely sloughed.

### EXAMPLE 6

Human Tumor Tissue Staining

An IHC study was undertaken to determine the cancer association of the 7BD-33-11A and 1A245.6 antigen with human breast cancers and whether either antibody was likely to recognize human cancers. A comparison was made for vimentin (positive control), and an antibody directed towards Aspergillus niger glucose oxidase, an enzyme which is neither present nor inducible in mammalian tissues (negative control). A breast cancer tissue array derived from 50 breast cancer patients and 10 samples derived from non-neoplastic breast tissue in breast cancer patients were used (Imgenex Corporation, San Diego, Calif.). The following information was provided for each patient: age, sex, and diagnosis. The procedure for IHC from Example 5 was followed. All antibodies were used at a working concentration of 5 μg/ml.

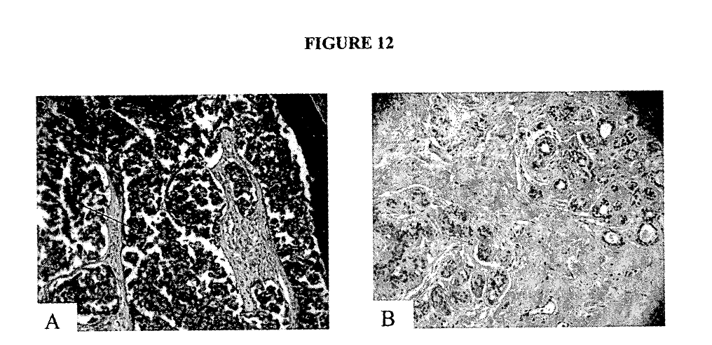

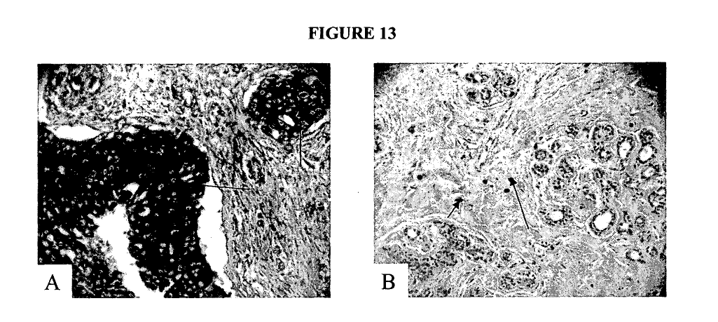

Table 2 provides a binding summary of 7BD-33-11A and 1A245.6 antibody staining of a breast cancer tissue array. Each array contained tumor samples from 50 individual patients. Overall, 36 percent of the 50 patients tested were positive for the 7BD-33-11A antigen (FIG. 12A) compared to 98 percent for 1A245.6 (FIG. 13A). For 7BD-33-11A, 0 out of 10 normal breast tissue samples from breast cancer patients were positive (FIG. 12B). Conversely, 9 out of 10 normal breast tissue samples were positive for 1A245.6. However, staining was due to infiltrating fibroblasts in the majority of cases (FIG. 13B). No correlation between estrogen and progesterone receptor status was evident for 1A245.6 (Table 3). There were a slightly higher number of positive 7BD-33-11A antigen tissues that were also estrogen and progesterone receptor expressers (Table 4). For the 7BD-33-11A antigen, it also appeared there was a trend to greater positive expression with higher tumor stage (Table 4) and for 1A245.6, the intensity of tissue staining appeared to correlate with higher tumor stage (Table 3). Both the 7BD-33-11A and 1A2425.6 staining was specific for cancerous cells and staining occurred on both the membrane and within the cytoplasm. The staining pattern, from both 7BD-33-11A and 1A245.6, showed that in patient samples, the antibody is highly specific for malignant cells and the respective antigens are present on the cell membrane thereby making it an attractive druggable target.

TABLE 2
IHC On Human Breast Tumor Tissue

| Sec. | | | | | |
|---|---|---|---|---|---|
| No. | Sex | Age | Diagnosis | 1A245.6 | 7BD-33-11A |
| 1 | F | 28 | Infiltrating duct carcinoma | +++ MC | ++ MC |
| 2 | F | 71 | Solid papillary carcinoma | +++ MC | +/− |
| 3 | F | 26 | Infiltrating duct carcinoma | ++ MC | − |
| 4 | F | 43 | Infiltrating duct carcinoma | ++ MC | +/− |
| 5 | F | 39 | Infiltrating duct carcinoma | + MC Tumor, +++ Necrotic area | +/− |
| 6 | F | 46 | Ductal carcinoma in situ | ++ MC | +/− |
| 7 | F | 47 | Infiltrating duct carcinoma | +++ MC Tumor, ++ stroma | + MC |
| 8 | M | 67 | Infiltrating duct carcinoma | +++ MC | + MC |
| 9 | F | 33 | Infiltrating duct carcinoma | ++ MC | − |
| 10 | F | 47 | Infiltrating duct carcinoma | ++ MC | − |
| 11 | F | 49 | Invasive lobular carcinoma | − Tumor, +/− Fibroblasts | − |
| 12 | F | 46 | Infiltrating duct carcinoma | +++ MC | − |
| 13 | F | 39 | Infiltrating duct carcinoma | ++ MC | − |
| 14 | F | 43 | Infiltrating lobular carcinoma | +++ MC | +/− |
| 15 | F | 54 | Infiltrating lobular carcinoma | ++ MC | +/− |
| 16 | F | 58 | Infiltrating duct carcinoma | ++ MC Tumor, Stroma, +++ Necrotic area | +/− |
| 17 | F | 37 | Infiltrating duct carcinoma | +++ MC | − |
| 18 | F | 43 | Infiltrating duct carcinoma | +++ MC Tumor, Stroma | +++ M/C |
| 19 | F | 51 | Infiltrating duct carcinoma | +++ MC | + MC |
| 20 | F | 80 | Medullary carcinoma | ++ MC | − |
| 21 | F | 36 | Infiltrating duct carcinoma | ++ MC Tumor, Stroma | − |
| 22 | F | 59 | Infiltrating duct carcinoma | + MC | +/− Blood vessels |
| 23 | F | 34 | Ductal carcinoma in situ | ++ MC Tumor, Stroma, +++ Necrotic area | + Tumor, |
| | | | | | ++ Necrotic area |
| 24 | F | 54 | Infiltrating duct carcinoma | ++ MC | +/− |
| 25 | F | 47 | Infiltrating duct carcinoma | +++ MC | ++ MC |
| 26 | F | 53 | Infiltrating duct carcinoma | ++ MC | − |
| 27 | F | 59 | Infiltrating duct carcinoma | + MCTumor, Stroma, Endothelium of blood vessels | +/− Necrotic area |
| 28 | F | 60 | Signet ring cell carcinoma | +++ MC | − |
| 29 | F | 37 | Infiltrating duct carcinoma | +++ MC | ++ MC |
| 30 | F | 46 | Infiltrating duct carcinoma | ++ MC | +/− |
| 31 | F | 35 | Infiltrating duct carcinoma | +/− | − |
| 32 | F | 47 | Infiltrating duct carcinoma | + Tumor, +++ Necrotic area | − |
| 33 | F | 54 | Infiltrating duct carcinoma | ++ MC | − |
| 34 | F | 47 | Infiltrating duct carcinoma | + MC Tumor, Stroma | − |
| 35 | F | 41 | Infiltrating duct carcinoma | +++ MC | − |
| 36 | F | 38 | Infiltrating duct carcinoma | +++ MC | − |
| 37 | F | 55 | Infiltrating duct carcinoma | + MC Tumor, Stroma | − |
| 38 | F | 65 | Infiltrating duct carcinoma | ++ MC Tumor, Stroma | − |
| 39 | M | 66 | Infiltrating duct carcinoma | + MC Tumor, Stroma | − |
| 40 | F | 44 | Infiltrating duct carcinoma | ++ MC | − |
| 41 | F | 52 | Metastatic carcinoma in lymph node | ++ MC | − |
| 42 | F | 32 | Metastatic carcinoma in lymph node | ++ MC | − |
| 43 | F | 58 | Metastatic carcinoma in lymph node | +++ MC | +/− |
| 44 | F | 52 | Metastatic carcinoma in lymph node | ++ MC | − |
| 45 | F | 58 | Metastatic carcinoma in lymph node | + MC | − |
| 46 | F | 38 | Metastatic carcinoma in lymph node | +++ MC | − |
| 47 | F | 45 | Metastatic carcinoma in lymph node | +/− | − |
| 48 | F | 45 | Metastatic carcinoma in lymph node | ++ MC | − |
| 49 | F | 29 | Metastatic carcinoma in lymph node | ++ MC | − |
| 50 | F | 61 | Metastatic carcinoma in lymph node | ++ MC | − |
| *51 | F | 46 | Nipple | ++ Sebaceous glands | − |
| *52 | F | 47 | Nipple | ++ MC | − |
| *53 | F | 40 | Normal Breast | − | − |
| *54 | F | 43 | Normal Breast | +/− Fibroblasts | − |
| *55 | F | 40 | Normal Breast | ++ Lobular epithelium, Fibroblasts, Endothelium | − |
| *56 | F | 40 | Normal Breast | +/− Fibroblasts | − |
| *57 | F | 45 | Normal Breast | +/− Fibroblasts | − |
| *58 | F | 44 | Normal Breast | +/− Fibroblasts | − |
| *59 | F | 37 | Normal Breast | ++ Lobular epithelium, Fibroblasts | − |
| *60 | F | 51 | Normal Breast | +/− Fibroblasts | − |

Abbreviations:
PS: the section is partially detached;
*Non-neoplastic breast tissue in breast cancer patient.

TABLE 3
IHC Correlation Summary For 1A245.6

| | | | Binding Score | | | | | | |
|---|---|---|---|---|---|---|---|---|---|
| | | Total # | − | +/− | + | ++ | +++ | Total positive | % positiveof total |
| PatientSamples | Tumor | 50 | 1 | 2 | 8 | 23 | 16 | 49 | 98% |
| | Normal | 10 | 1 | 5(Fibroblasts) | 0 | 4 | 0 | 9 | 90% |
| ER | ER+ | 28 | 0 | 1 | 2 | 14 | 11 | 28 | 100% |
| Status | ER− | 22 | 1 | 1 | 6 | 9 | 5 | 21 | 96% |
| | Unknown | 0 | 0 | 0 | 0 | 0 | 0 | 0 | 0% |
| PR Status | PR+ | 19 | 0 | 0 | 1 | 8 | 10 | 19 | 100% |
| | PR− | 30 | 1 | 2 | 7 | 14 | 6 | 29 | 97% |
| | Unknown | 1 | 0 | 0 | 0 | 0 | 0 | 1 | 100% |
| AJCCTumorStage | T1 | 4 | 0 | 1 | 1 | 1 | 1 | 4 | 100% |
| | T2 | 21 | 1 | 0 | 6 | 9 | 5 | 20 | 95% |
| | T3 | 20 | 0 | 1 | 1 | 10 | 8 | 20 | 100% |
| | T4 | 5 | 0 | 0 | 0 | 3 | 2 | 5 | 100% |

TABLE 4
IHC Correlation Summary For 7BD-33-11A

| | | | Binding Score | | | | | | |
|---|---|---|---|---|---|---|---|---|---|
| | | Total # | − | +/− | + | ++ | +++ | Total positive | % positiveof total |
| PatientSamples | Tumor | 50 | 30 | 12 | 4 | 3 | 1 | 20 | 40% |
| | Normal | 10 | 10 | 0 | 0 | 0 | 0 | 0 | 0% |
| ER Status | ER+ | 28 | 16 | 9 | 1 | 2 | 0 | 12 | 43% |
| | ER− | 22 | 15 | 3 | 2 | 1 | 1 | 7 | 32% |
| | Unknown | 0 | 0 | 0 | 0 | 0 | 0 | 0 | 0% |
| PR Status | PR+ | 19 | 9 | 6 | 2 | 2 | 0 | 10 | 53% |
| | PR− | 30 | 20 | 6 | 2 | 1 | 1 | 10 | 33% |
| | Unknown | 1 | 1 | 0 | 0 | 0 | 0 | 0 | 0% |
| AJCC Tumor Stage | T1 | 4 | 4 | 0 | 0 | 0 | 0 | 0 | 0% |
| | T2 | 21 | 14 | 3 | 2 | 1 | 1 | 7 | 33% |
| | T3 | 20 | 11 | 6 | 2 | 1 | 0 | 9 | 45% |
| | T4 | 5 | 1 | 3 | 0 | 1 | 0 | 4 | 80% |

To determine whether either the 7BD-33-11A or 1A245.6 antigen is expressed on other human cancer tissues in addition to breast cancer, both antibodies were individually tested on a multiple human tumor tissue array (Intgenex, San Diego, Calif.). The following information was provided for each patient: age, sex, organ and diagnosis. The staining procedure used was the same as the one outlined in Example 5. Vimentin was used as a positive control antibody and the same negative control antibody was used as described for the human breast tumor tissue array. All antibodies were used at a working concentration of 5 μg/mL.

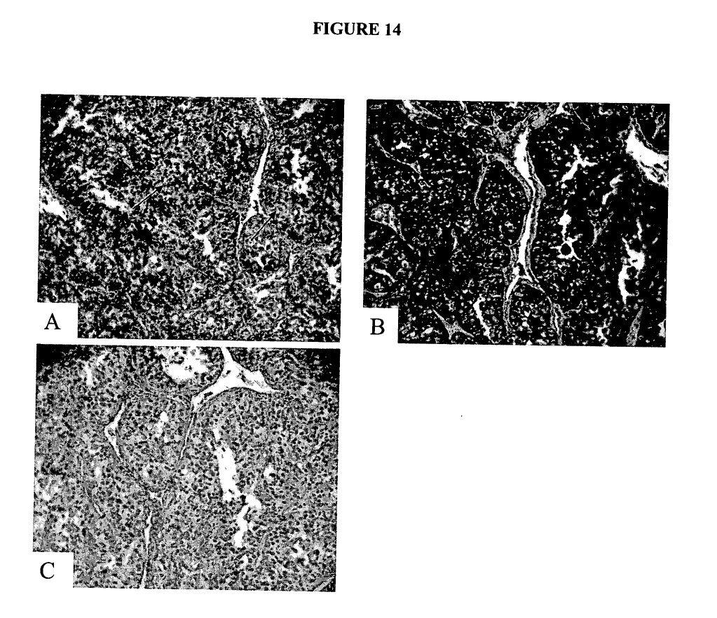

As outlined in Table 5, 7BD-33-11A stained a number of various human cancers besides breast. The following tumor types were positive for 7BD-33-11A: skin (1/2), lung (3/4), liver (2/3), stomach (4/5), thyroid (2/2), prostate (1/1), uterus (4/4) and kidney (3/3) (FIG. 14A). Several other tumor types also occasionally stained positive. Other tumor tissues were negative for 7BD-33-11A expression; ovary (0/3), testis (0/1), brain (0/2) and lymph node (0/2). Conversely, 1A245.6 stained every tumor tissue type tested. However, some of the strongest staining was seen on malignant cells of the skin, lung, liver, uterus, kidney (FIG. 14B), stomach and bladder. As seen with the breast cancers, 7BD-33-11A and 1A245.6 staining was localized on the membrane and within the cytoplasm of cancerous cells.

Therefore, it appears that the 7BD-33-11A and 1A245.6 antigen is not solely found on the membranes of breast cancers but also on the membrane of a large variety of tumor types including prostate. These results indicate that both 7BD-33-11A and 1A245.6 have potential as a therapeutic drug in a wide variety of tumor types in addition to breast and prostate cancer.

TABLE 5
IHC On Human Multi-Tumor Tissue Array

| Sec. | | | | | | |
|---|---|---|---|---|---|---|
| No. | Age | Sex | Organ | Diagnosis | 1A245.6 | 7BD-33-11A |
| 1 | 59 | M | Skin | Malignant melanoma | ++ M/C | ++ M/C |
| 2 | 25 | F | Skin | SSC | ++ M/C | − |
| 3 | 50 | F | Breast | Infiltrating ductal carcinoma | ++ M/C | ++ M/C |
| 4 | 57 | F | Breast | Invasive papillary carcinoma | + M/C | − |
| 5 | 35 | F | Breast | Infiltrating lobular carcinoma | + M/C | F |
| 6 | 40 | M | Lymph node | Malignant lymphoma, immunoplatic | + M/C | − |
| 7 | 58 | M | Lymph node | Metastatic adenoca from stomach | + M/C | − |
| 8 | 53 | F | Bone | Osteosarcoma | ++ M/C | ++ M/C |
| 9 | 26 | M | Bone | Giant cell tumor | ++ M/C | − |
| 10 | 40 | M | Bone | Chondro sarcoma | CS | CS |
| 11 | 51 | F | Soft tissue | Liposarcoma | +/ − | − |
| 12 | 47 | F | Soft tissue | Neuro fibromatosis | +/− | − |
| 13 | 74 | M | Nasalcavity | Inverted papilloma | + M/C | +/− |
| 14 | 57 | M | Larynx | SCC | + Tumor, ++ Lymphocytes, Stroma | +/− |
| 15 | 60 | M | Lung | Adenocarcinoma | +++ M/C | ++ M/C |
| 16 | 51 | F | Lung | SCC | ++ M/C | + M/C |
| 17 | 68 | F | Lung | Adenocarcinoma | + M/C | − |
| 18 | 60 | M | Lung | Small cell carcinoma | ++ M/C | + M/C |
| 19 | 88 | F | Tongue | SCC | + M/C | +/− |
| 20 | 34 | F | Parotid gland | Pleomorphic adenoma | +/− mucin | − |
| 21 | 50 | F | Parotid gland | Warthin tumor | +++ M/C | +++ M/C |
| 22 | 40 | F | Parotid gland | Pleomorphic adenoma | + M/C | +/− |
| 23 | 56 | M | Sub mandibular gland | Salivary duct carcinoma | + M/C | − |
| 24 | 69 | F | Liver | Cholangiocarcinoma | +++ M/C | ++ M/C |
| 25 | 51 | M | Liver | Metastatic gastric Ca. | ++ M/C | − |
| 26 | 64 | M | Liver | HCC | +++ M/C | ++ M/C |
| 27 | 62 | F | Gallbladder | Adenocarcinoma | ++ M/C | + M/C |
| 28 | 64 | F | Pancreas | Adenocarcinoma | ++ M/C | ++ M/C |
| 29 | 68 | M | Esophagus | SCC | + M/C | +/− |
| 30 | 73 | M | Stomach | Adenocarcinoma (poorly differentiated) | ++ M/C | + M/C |
| 31 | 63 | M | Stomach | Adenocarcinoma (moderately differentiated) | ++ M/C | +/− |
| 32 | 59 | F | Stomach | Signet ring cell carcinoma | ++ M/C | +/− |
| 33 | 62 | M | Stomach | Malignant Lymphoma | + M/C | + Blood vessels |
| 34 | 51 | M | Stomach | Border line stromal tumor | +++ M/C | + M/C |
| 35 | 42 | M | Small Intestine | Malignant stromal tumor | ++ M/C | − |
| 36 | 52 | F | Appendix | Pseuomyxoma peritonia | − PS | − |
| 37 | 53 | M | Colon | Adenocarcinoma | + M/C | +/− |
| 38 | 67 | M | Rectum | Adenocarcinoma | ++ M/C | − |
| 39 | 75 | F | Kidney | Transitional cell carcinoma | +++ M/C | ++ M/C |
| 40 | 54 | F | Kidney | Renal cell carcinoma | ++ M/C | +/− |
| 41 | 75 | F | Kidney | Renal cell carcinoma | +++ M/C | ++ M/C |
| 42 | 65 | M | Urinary bladder | Carcinoma (poorly differentiated) | ++ M/C | − |
| 43 | 67 | M | Urinary bladder | Transitional cell carcinoma (high grade) | +++ M/C | +++ M/C |
| 44 | 62 | M | Prostate | Adenocarcinoma | + M/C | +/− |
| 45 | 30 | M | Testis | Seminoma | + M/C | − |
| 46 | 68 | F | Uterus | Endometrial adenocarcinoma | +++ M/C | ++ M/C |
| 47 | 57 | F | Uterus | Leimyosacoma | + C | +/− |
| 48 | 45 | F | Uterus | Leiomyoma | ++ C | +/− |
| 49 | 63 | F | Uterine cervix | SCC | + Tumor, ++ Stroma, Lymphocytes | +/− |
| 50 | 12 | F | Ovary | Endodermal sinus tumor | ++ M/C | − |
| 51 | 33 | F | Ovary | Mucinous adenocarcinoma | ++ M/C | − |
| 52 | 70 | F | Ovary | Fibrothecoma | ++ M/C | − |
| 53 | 67 | F | Adrenal gland | Cortical carcinoma | ++ M/C | + M/C |
| 54 | 61 | F | Adrenal gland | Pheohromcytoma | +++ M/C | − |
| 55 | 54 | M | Thyroid | Papillary carcinoma | +++ M/C | ++ M/C |
| 56 | 58 | F | Thyroid | Minimally invasive follicullar carcinoma | ++ M/C | + M/C |
| 57 | 74 | M | Thymus | Thymoma | + C | +/− |
| 58 | 66 | F | Brain | Meningioma | ++ M/C | − |
| 59 | 62 | M | Brain | Glioblastoma multiforme | + M/C | − |

Abbreviations:
M: Membrane staining;
C: Cytoplasmic staining;
M/C: Membrane-cytoplasmic staining;
CS: the section is completely sloughed;
PS: the section is partially sloughed;
F: The section is folded;
SSC: Squamous cell carcinoma;
HCC: Hepatocellular carcinoma.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objects and obtain the ends and advantages mentioned, as well as those inherent therein. Any oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of extending survival and delaying disease progression by treating a human tumor in a mammal, wherein said tumor expresses an antigen which specifically binds to a monoclonal antibody or antigen binding fragment thereof which has the identifying characteristics of a monoclonal antibody encoded by a clone deposited with the ATCC as accession number PTA-4890 comprising administering to said mammal said monoclonal antibody in an amount effective to reduce said mammal's tumor burden, whereby disease progression is delayed and survival is extended.

2. The method of claim 1 wherein said antibody is conjugated to a cytotoxic moiety.

3. The method of claim 2 wherein said cytotoxic moiety is a radioactive isotope.

4. The method of claim 1 wherein said antibody activates complement.

5. The method of claim 1 wherein said antibody mediates antibody dependent cellular cytotoxicity.

6. The method of claim 1 wherein said antibody is a murine antibody.

7. The method of claim 1 wherein said antibody is a humanized antibody

8. The method of claim 1 wherein said antibody is a chimerized antibody.

9. An isolated monoclonal antibody or antigen binding fragments thereof encoded by the clone deposited with the ATCC as PTA-4890.

10. The isolated antibody or antigen binding fragments of claim 9, wherein said isolated antibody or antigen binding fragments thereof is humanized.

11. The isolated antibody or antigen binding fragments of claim 9 conjugated with a member selected from the group consisting of cytotoxic moieties, enzymes, radioactive compounds, and hematogenous cells.

12. The isolated antibody or antigen binding fragments of claim 9, wherein said isolated antibody or antigen binding fragments thereof is a chimerized antibody.

13. The isolated antibody or antigen binding fragments of claim 9, wherein said isolated antibody or antigen binding fragments thereof is a murine antibody.

14. The isolated clone deposited with the ATCC as PTA-4890.

15. A binding assay to determine presence of cancerous cells in a tissue sample selected from a human tumor comprising:
providing a tissue sample from said human tumor;
providing an isolated monoclonal antibody or antigen binding fragment thereof encoded by the clone deposited with the ATCC as PTA-4890;
contacting said isolated monoclonal antibody or antigen binding fragment thereof with said tissue sample; and
determining binding of said isolated monoclonal antibody or antigen binding fragment thereof with said tissue sample;
whereby the presence of said cancerous cells in said tissue sample is indicated.

16. The binding assay of claim 15 wherein the human tumor tissue sample is obtained from a tumor originating in a tissue selected from the group consisting of colon, ovarian, lung, prostate and breast tissue.

17. A process of isolating or screening for cancerous cells in a tissue sample selected from a human tumor comprising:
providing a tissue sample from a said human tumor;
providing an isolated monoclonal antibody or antigen binding fragment thereof encoded by the clone deposited with the ATCC as PTA-4890;
contacting said isolated monoclonal antibody or antigen binding fragment thereof with said tissue sample; and
determining binding of said isolated monoclonal antibody or antigen binding fragment thereof with said tissue sample;
whereby said cancerous cells are isolated by said binding and their presence in said tissue sample is confirmed.

18. The process of claim 17 wherein the human tumor tissue sample is obtained from a tumor originating in a tissue selected from the group consisting of colon, ovarian, lung, prostate and breast tissue.

19. A method of extending survival and delaying disease progression by treating a human tumor in a mammal, wherein said tumor expresses an antigen which specifically binds to a monoclonal antibody or antigen binding fragment thereof which has the identifying characteristics of a monoclonal antibody encoded by a clone deposited with the ATCC as accession number PTA-4889 comprising administering to said mammal said monoclonal antibody in an amount effective to reduce said mammal's tumor burden, whereby disease progression is delayed and survival is extended.

20. The method of claim 19 wherein said antibody is conjugated to a cytotoxic moiety.

21. The method of claim 20 wherein said cytotoxic moiety is a radioactive isotope.

22. The method of claim 19 wherein said antibody activates complement.

23. The method of claim 19 wherein said antibody mediates antibody dependent cellular cytotoxicity.

24. The method of claim 19 wherein said antibody is a murine antibody.

25. The method of claim 19 wherein said antibody is a humanized antibody

26. The method of claim 19 wherein said antibody is a chimerized antibody.

27. An isolated monoclonal antibody or antigen binding fragments thereof encoded by the clone deposited with the ATCC as PTA-4889.

28. The isolated antibody or antigen binding fragments of claim 27, wherein said isolated antibody or antigen binding fragments thereof is humanized.

29. The isolated antibody or antigen binding fragments of claim 27 conjugated with a member selected from the group consisting of cytotoxic moieties, enzymes, radioactive compounds, and hematogenous cells.

30. The isolated antibody or antigen binding fragments of claim 27, wherein said isolated antibody or antigen binding fragments thereof is a chimerized antibody.

31. The isolated antibody or antigen binding fragments of claim 27, wherein said isolated antibody or antigen binding fragments thereof is a murine antibody.

32. The isolated clone deposited with the ATCC as PTA-4889.

33. A binding assay to determine presence of cancerous cells in a tissue sample selected from a human tumor comprising:
providing a tissue sample from said human tumor;
providing an isolated monoclonal antibody or antigen binding fragment thereof encoded by the clone deposited with the ATCC as PTA-4889;
contacting said isolated monoclonal antibody or antigen binding fragment thereof with said tissue sample; and
determining binding of said isolated monoclonal antibody or antigen binding fragment thereof with said tissue sample;
whereby the presence of said cancerous cells in said tissue sample is indicated.

34. The binding assay of claim 33 wherein the human tumor tissue sample is obtained from a tumor originating in a tissue selected from the group consisting of colon, ovarian, lung, prostate and breast tissue.

35. A process of isolating or screening for cancerous cells in a tissue sample selected from a human tumor comprising:
providing a tissue sample from a said human tumor;
providing an isolated monoclonal antibody or antigen binding fragment thereof encoded by the clone deposited with the ATCC as PTA-4889;
contacting said isolated monoclonal antibody or antigen binding fragment thereof with said tissue sample; and
determining binding of said isolated monoclonal antibody or antigen binding fragment thereof with said tissue sample;
whereby said cancerous cells are isolated by said binding and their presence in said tissue sample is confirmed.

36. The process of claim 35 wherein the human tumor tissue sample is obtained from a tumor originating in a tissue selected from the group consisting of colon, ovarian, lung, prostate and breast tissue.
